(19) United States
(12) Patent Application Publication
(10) Pub. No.: US 2018/0261334 A1
(43) Pub. Date: Sep. 13, 2018

(54) Communication System, A Mobile Phone And An Informing Unit

(71) Applicant: Masahide Tanaka, Osaka (JP)

(72) Inventor: Masahide Tanaka, Osaka (JP)

(73) Assignee: NL Giken Incorporated, Osaka (JP)

(21) Appl. No.: 15/978,349

(22) Filed: May 14, 2018

Related U.S. Application Data
(63) Continuation of application No. 14/932,972, filed on Nov. 5, 2015.
Application No. 14/932,972 is a continuation of application No. 12/838,504, filed on Jul. 19, 2010, now abandoned.
Application No. 12/838,504 is a continuation-in-part of application No. 12/576,245, filed on Oct. 9, 2009, now Pat. No. 8,680,408.

(30) Foreign Application Priority Data
Jul. 25, 2009 (JP) 2009-173774

(51) Int. Cl. G16H 80/00; G01G 23/37; H04W 24/08; G01G 19/44; H04W 88/06

(52) U.S. Cl. CPC G16H 80/00; G01G 23/3735; H04W 88/06; G01G 19/44; G01G 23/3728; H04W 24/08

(57) ABSTRACT

A mobile phone capable of communication in a local area comprises a radiowave telecommunication function, an infrared wireless communication function, a processor for processing data due to the infrared wireless communication function in accordance with a software downloaded by the radiowave telecommunication function, and a memory for the downloaded software. A display is also provided to display the data due to the infrared wireless communication function as well the processed data in accordance with the software downloaded and stored in the memory. A data input portion in accordance with the downloaded software is further provided, the input data being also displayed. A controller is provided to download the software from an outside server through internet by means of the radiowave receiver. The software includes a guidance how to deal with such a situation that the infrared transmitter fails in the wireless communication within the local area.

### CROSS RELATION TO RELATED APPLICATION

This application is a Continuation Application of U.S. application Ser. No. 14/932,972 filed Nov. 5, 2015, which is a continuation of U.S. application Ser. No. 12/838,504 filed Jul. 19, 2010, which is a continuation-in-part of U.S. application Ser. No. 12/576,245, filed Oct. 9, 2009, herein incorporated by reference in its entirety.

### BACKGROUND OF THE INVENTION

### 1. Field of the Invention

This invention relates to a communication system.

### 2. Description of the Related Art

In this field of the art, various attempts have been done with respect to a communication system. For example, Japanese Laid-open Patent Application No. 2000-285178 proposes an electronic mother-child notebook having health information linked with a treatment database of a medical institute. Japanese Laid-open Patent Application No. 2005-190387 proposes another electronic mother-child notebook of a card memory type, the data of which is retrieved and displayed in the form of a growth chart on a user terminal by means of a browser provided by a server through the internet. On the other hand, Japanese Laid-open Patent Application No. 2006-253802 proposes a plug attachable to a mobile phone to disabling radiowave transmitter and to inform other people of the mobile phone functioning with transmitter disabled.

However, there still exist in this field of art many demands for improvements of a communication system.

### SUMMARY OF THE INVENTION

Preferred embodiments of this invention provide a communication system in a local area where radiowave transmission is inhibited.

Preferred embodiments of this invention provide a mobile phone capable of being used in a local area where radiowave transmission is inhibited.

Still more preferred embodiments of this invention provide an informing unit in a local area where radiowave transmission is inhibited.

Further more preferred embodiments of this invention provide a communication system suitable for use in a maternity hospital.

A preferred embodiment of this invention provides a communication system in a local area where radiowave transmission is inhibited comprising a combination of a mobile phone and a receiving system. The mobile phone includes a first transmitter arranged to generate radiowave used in a telecommunication function, a second transmitter capable of a wireless communication permitted in the local area with the first transmitter is disabled, and a data input portion arranged to input data to be transmitted by the second transmitter. On the other hand, the receiving system is located within the local area and includes a receiver arranged to receive the transmission data from the mobile phone by means of the second transmitter, and a processor arranged to process the received data.

In a detailed design according to the above preferred embodiment, the communication system further comprises an informing unit including a rediowave sensor and an indicator arranged to continue indication of no radiowave transmission as long as the radiowave sensor does not detect radiowave. In the above design, indication is positively made when the detection by the radiowave sensor is negative. By means of normally making indication of safety according to the above design, notification effect is increased for the purpose of removing misunderstanding of the surrounding people, which otherwise may cause troubles.

According to still detailed design, the informing unit is arranged to be attachable to the mobile phone within the local area under suitable administration in the local area. According to another still detailed design, the informing unit is included in the receiving system as an equipment of the local area in cooperation with the mobile phone.

A typical example of the local area is a maternity hospital and the processor in the receiving system is arranged to process newborn data inputted by a mother at the mobile phone and transmitted though the second transmitter. In detail, the newborn data relates to at least one of body temperature, defecation, urination, breast-feed and disgorgement.

Another preferred embodiment of this invention provides a mobile phone capable of being used in a local area where radiowave transmission is inhibited. The mobile phone comprises a first communicator arranged to generate radiowave used in a telecommunication function, a selector arranged to selectively switch the mobile communication device between a first mode of functioning in which the first communication is enabled, and a second mode of functioning in which the first communicator is disabled, a second communicator arranged to function in the second mode by means of a wireless communication permitted in the local area, a display arranged to function in the second mode to display data communicated through the second communicator; and a notification unit arranged to inform surrounding people of the fact that the mobile communication device is used in the second mode. In a detailed design, the second communicator includes a transmitter arranged to transmit data displayed on the display. In another detailed design, the second communicator includes a receiver arranged to receive data to be displayed on the display. According to the preferred embodiment, data communication in the local area, such as information exchange relating to a newborn in a maternity hospital, is advantageously possible without misunderstanding of surrounding people.

Still another preferred embodiment of this invention provides an informing unit in a local area where radiowave transmission is inhibited. The informing unit comprises a rediowave sensor, and an indictor arranged to continue indication of no radiowave transmission as long as the radiowave sensor does not detect radiowave.

In a detailed design according to the above preferred embodiment, the informing unit is arranged to be attachable to the mobile phone within the local area As a typical example of still detailed design, the informing unit is formed as a strap to be attached to the mobile phone and detached from the mobile phone under strict administration rule in the local area for removing confusion.

According to another detailed design, the informing unit further comprises an own power source. In this manner, it is possible to positively make indication when the detection by the radiowave sensor is negative. Thus, it is possible to continue the safety indication for increasing the notification effect to remove misunderstanding of the surrounding people, which otherwise may cause troubles.

According to still another detailed design, the informing unit further comprises a second indictor arranged to make a caution if the radiowave sensor detects radiowave. By means of providing the dual indicators in this manner, one of the indicators complementary makes an indication for increasing notification effect as long as the radiowave sensor works. In this detailed design, it is possible to arrange the second indicator to be powered by the radiowave sensor detecting radiowave in case of a battery power source is exhausted.

According to another detailed design, the informing unit is equipped within the local area. In more detail, the informing unit is possible to be advantageously located at a key point where a wireless communication permitted in the local area is made to remove misunderstanding of the surrounding people close to the key point.

Other features, elements, arrangements, steps, characteristics and advantages according to this invention will be readily understood from the detailed description of the preferred embodiments in conjunction with the accompanying drawings.

The above description should not be deemed to limit the scope of this invention, which should be properly determined on the basis of the attached claims.

### BRIEF DESCRIPTION OF THE DRAWINGS

FIG. 1 is a block diagram showing a mother-child database system including a mobile phone, an electronic scale and a mother-child database server according to Embodiment 1 of this invention.

FIG. 2 is a graph showing a relationship between weight on a weight sensor and the sensor output in the electronic scale according to Embodiment 1 of this invention.

FIG. 3 is an example of display with the mobile phone functioning as an electronic mother-child notebook, in which a weight growth chart is displayed.

FIG. 4 is a flowchart showing the function carried out by a scale controller of the electronic scale in FIG. 1.

FIG. 5 is a flowchart showing a partial function carried out by a mobile phone controller of the mobile phone in FIG. 1 in relation to a mother-child notebook software.

FIG. 6 is a flowchart showing the mother-child notebook software function carried out by a server controller of the mother-child database server in FIG. 1.

FIG. 7 is a block diagram showing a mother-child database system including a mobile phone, a mother-child notebook control system in maternity hospital, a green strap, and a mother-child database server according to Embodiment 2 of this invention

FIG. 8 is a flowchart showing a partial function carried out by a mobile phone controller of mobile phone in FIG. 7 in relation to a mother-child notebook software.

### DETAILED DESCRIPTION OF THE PREFERRED EMBODIMENTS

### Embodiment 1

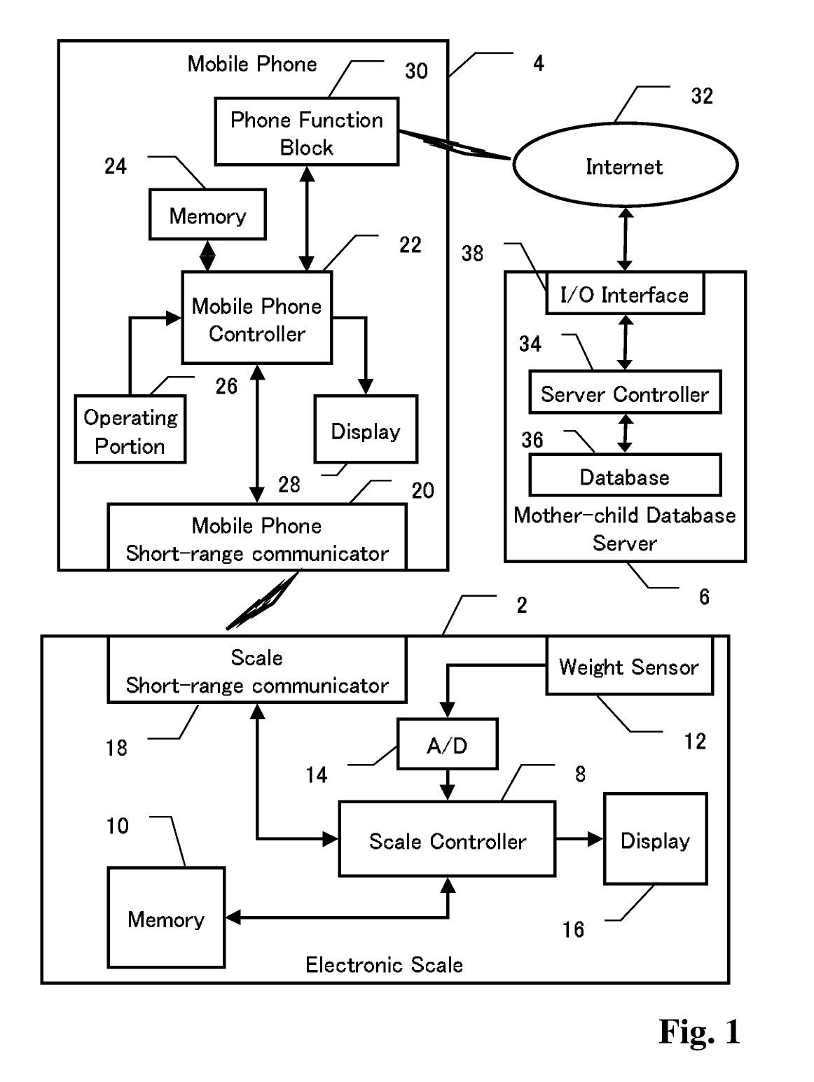

FIG. 1 is a block diagram showing the mother-child database system according to Embodiment 1 of this invention. The system in Embodiment 1 includes electronic scale 2, mobile phone 4 and mother-child database server 6. Electronic scale 2 is controlled by scale controller 8 having a micro computer. Memory 10 stores computer programs for the operation of scale controller 8 and also weight data which is to be gotten by weight sensor 12 and inputted through A/D converter 14.

The basic method of measuring weights of a mother and an infant by means of electronic scale is as follows. At first, if the mother alone steps on electronic scale 2, her weight is sensed by weight sensor 12 and sent to scale controller 8 by way of A/D converter 14, which is then stored in memory 10. Next, the mother with the infant in her arms steps on electronic scale 2. And, the weight of the mother plus the infant is sensed by weight sensor 12 in this time and sent to scale controller 8. Scale controller 8 subtracts the weight of mother alone form the total weight of mother plus infant to calculate the weight of the infant alone, which is stored in memory 10. Thus, the weights of mother alone and the infant alone are measured and stored, respectively.

Weight sensor 12 for electronic scale 2 in this invention for measuring weights of mother and infant according to the basic method above preferably consists of load cells for example and is capable of functioning both in a normal region and in a high sensitivity measurement region in which the output of weight sensor 12 more sensitively changes against the change in load thereon than in the normal region. By means of this feature, electronic scale 2 covers a wide dynamic range in total with the fine measurement region partially included. The fine measurement region, i.e., the high sensitivity measurement region both covers an assumed weight of mother alone and an assumed weight of mother plus infant so that the difference between the weights, which means the weight of infant, may be measured more precisely than in the normal region. In conformity with the fine measurement ability, the weight of infant calculated by the subtraction is stored in memory 10 with a scale interval finer than in weights measured in the normal region. Similarly, the weight of infant is displayed at display 16 with a unit finer than in weights measured in the normal region. For example, if the weight measured in the normal region is displayed in unit by 100 g, e.g., 46.8 kg, the calculated weight of infant is displayed in unit by 50 g, e.g., 3250 g.

The fine measurement region can be shifted by means of zero point adjustment of electronic scale 2. In more detail, when the weight of mother alone dips from the lower end of the fine measurement region, the zero point as the standard of sensor output is altered toward the positive side to shift the entire dynamic range in parallel toward the light weight side so that the fine measurement region may become to cover the weight of mother alone. On the other hand, when the weight of mother plus infant outweighs the upper end of the fine measurement region due to the weight of mother alone, the zero point is altered toward the negative side to shift the entire dynamic range in parallel toward the heavy weight side so that the fine measurement region may become to cover the weight of mother plus infant Since the weight of infant alone is the relative difference between the weight of mother alone and the weight of mother plus infant, which does not depend on the weight of mother alone, the weight of infant alone can be calculated independently from the degree of zero point adjustment. However, the degree or amount of zero point adjustment should be recorded in memory 10 for the purpose of correcting the record of the weight of mother alone when the zero point adjustment is made.

The weights of mother alone and infant alone measured and stored in accordance with the above manner, is to be retrieved from memory 10 so as to be sent from scale short-range communicator 18 to mobile phone short-range communicator 20. As short-range communicators 18 and 20, wireless local area network (LAN) or infrared data communication system or the like is applicable. Mobile phone controller 22 controls memory 24 to store the weight data received from electronic scale 2. The data of infant weight is retrieved in response to the operation at operating portion 26 and displayed at display 28. The weight data on display 28 is in a form of growth chart showing the monthly change in weight of infant since its birth.

Mobile phone controller 22 having a micro computer functions in accordance with computer program stored in memory 24 to originally and mainly control the entire mobile phone 4 by means of phone function block 30. Operation portion 26 and display 28 are also originally and mainly arranged for the functions of mobile phone 4 carried out by phone function block 30. Operation portion 26 and display 28 are, however, advantageously utilized for displaying growth chart of infant on the basis of weight data from electronic scale 2. Memory 24 of mobile phone 4 can also store various additional data relating to the growth of infant such as body height and circumference of head. On the basis of such additional data in memory 24, display 28 can similarly show a growth chart for body height and circumference of head of infant since its birth.

Memory 24 of mobile phone 4 can further store prehistory data of expectant mother toward birthing Various data above relating to mother and infant can be totally treated by mother-child notebook software installed in memory 24. Thus mobile phone 4 serves as electronic mother-child notebook. The mother-child notebook software is, for example, provided when an expectant mother applies for the mother-child notebook governed by a public institution. Upon provision of the mother-child notebook software, the expectant mother is informed under a strict personal information protection procedure a mother-child notebook identification number (ID) and tentative password for accessing to mother-child database server 6.

Phone function block 30 makes it possible for mobile phone 4 to communicate with mother-child database server 6 by way of internet 32. Mother-child database server 6 including server controller 34, database 36 and Input/Output interface 38 serves as an online portal for free information service in cooperation with healthcare authority and baby item industry. Thus, mobile phone 4 with the ID and password registered can login and receive from mother-child database server 6 a growth chart software with general data of growth curves for 10 percentile and 90 percentile on the basis of the latest year statistics to see how own baby measures up against other babies in height, weight, and head size. Further, if mobile phone 4 login with the ID and password to upload under anonymity the newest weight data in memory 24 for own baby, mother-child database server 6 provides mobile phone 4 with special data of growth curves for 10 percentile and 90 percentile created by means oft he statistic on the basis of the newest data currently uploaded under the same condition from a great number of mobile phones including mobile phone 4. The uploaded information is treated as anonymous weight data for the sole purpose of statistics with ID and password instantly deleted so as to strictly prevent any privacy from falling into the wrong hands. Further, for the incentive of uploading the newest anonymous weight data to increasing the reliability of statistics, a mobile phone uploading the weight data is entitled to enter into a private child-care Q&A corner provided by mother-child database server 6.

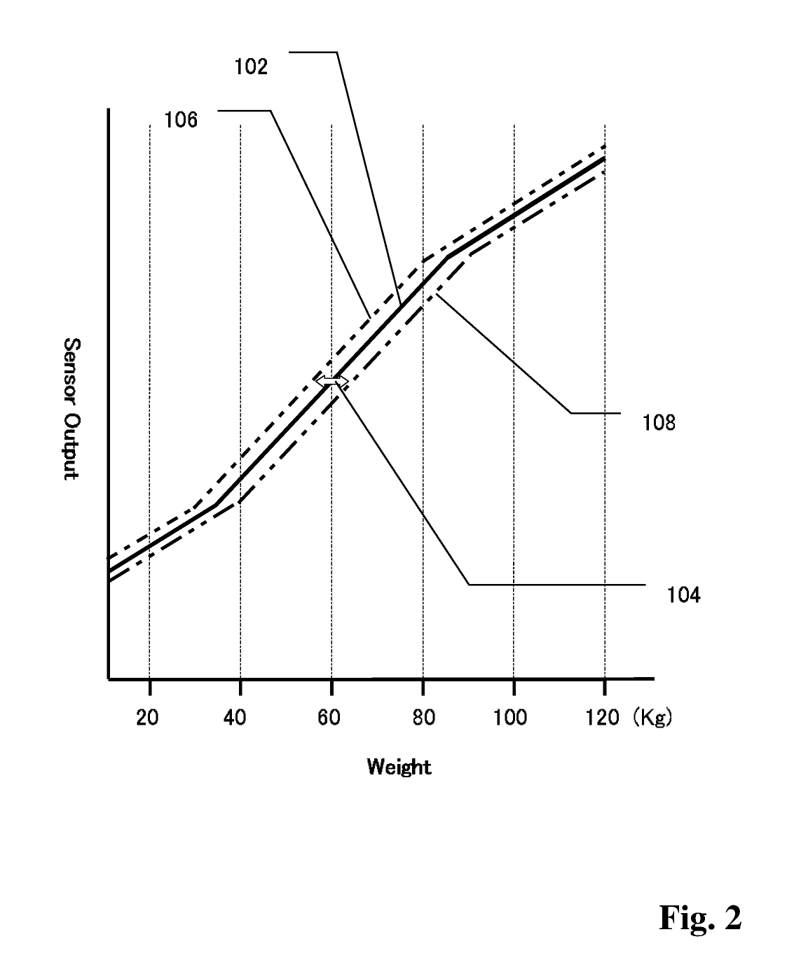

FIG. 2 is a graph showing a relationship between weight on weight sensor 12 and the sensor output with circuit compensation having been done. Curve 102 represents a standard curve with the zero point adjustment is set to the standard, curve 102 covers a range of weight form 20 kg to 120 kg. In the case of Curve 102, the high sensitivity measurement region spans from 35 kg to 85 kg, in which the change in sensor output against the change in weight on weight sensor 12 is greater than that in the normal regions on both side of the high sensitivity measurement region. Thus, a measurement in wide dynamic range in total from 20 kg to 120 kg as well as fine measurement with a finer scale interval in the partial range from 35 kg to 85 kg is possible.

Curve 102 can be shifted in parallel along a horizontal direction denoted by white allow 104 to shift the high sensitivity region toward the lighter weight side or the heavier weight side. Curve 106 represents a case that curve 102 is shifted toward the lighter weight side by means of shifting zero point toward the plus side, in which the high sensitivity region comes to span from 30 kg to 80 kg. On the other hand, curve 108 represents a case that curve 102 is shifted toward the heavier weight side by means of shifting zero point toward the minus side, in which the high sensitivity region comes to span from 40 kg to 90 kg. The width and the gradient of the high sensitivity region can be suitably determined with the condition of designing weight sensor 12, the requirement for the accuracy of measurement, the variability of expected weight of users and the adjustable range of zero point taken into consideration. If electronic scale 2 is not necessarily designed to make it freely available to the general public, but is designed only for use by a group of specifically assumed users such as in semi-custom design, the width of the high sensitivity region may be relatively narrow and also the zero point adjustment may be unnecessary. Further, in such a more simplified case that the dynamic range in total can be narrow due to a very limited user(s) assumed, the entire dynamic range may be designed as the high sensitivity region and mere the scale interval for displaying the weight of the infant is changed to be finer than in the case of displaying the weight of the mother.

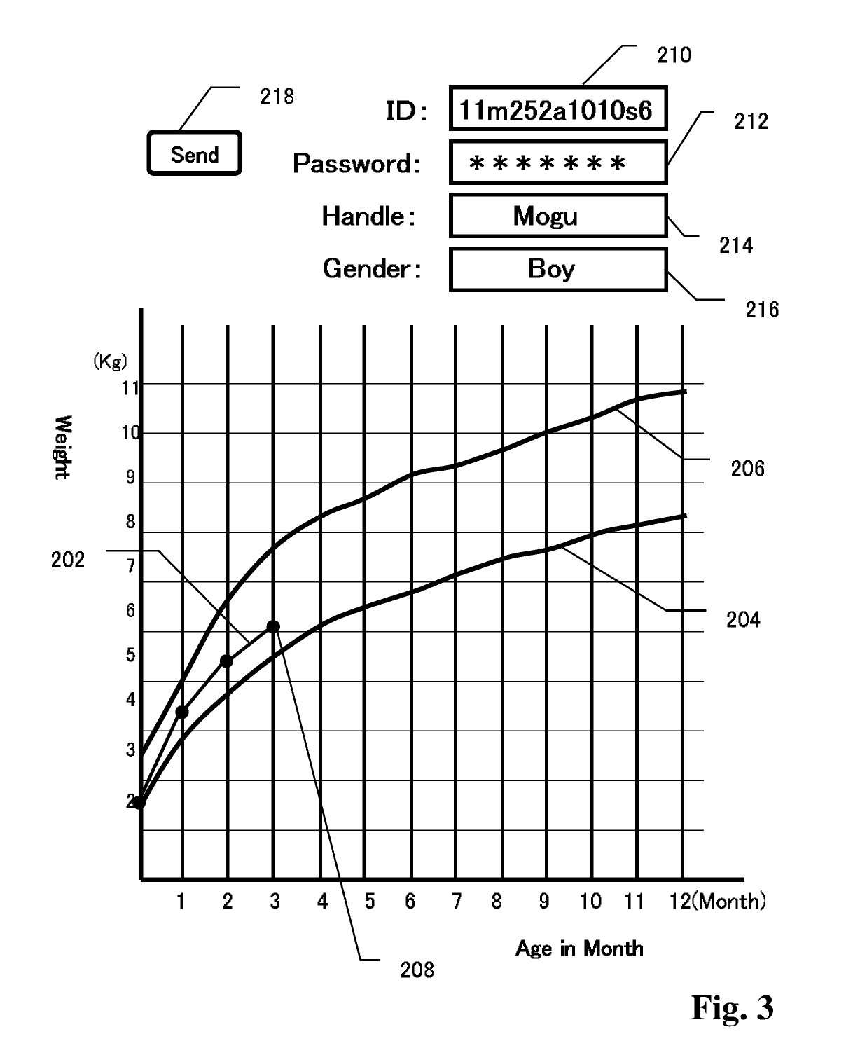

FIG. 3 is an example of display with mobile phone 4 functions as electronic mother-child notebook, in which a weight growth chart is displayed. The weight growth chart is processed by mother-child notebook software downloaded from mother-child database server 6 upon access with ID and password and installed in memory 24. The weight growth chart shows growth curve 202 which is created by automatically plotting the history of growth in weight stored in memory 24 on the graph of age in month vs. weight and automatically connecting the plots. Since the process of creating curve 202 is carried out by mobile phone controller 22 within mobile phone 4, data retrieved from memory 24 for plotting the graph does not leak from mobile phone 4.

In FIG. 3, the newest statistical both growth curve 204 representative of 10 percentile and the newest statistical growth curve 206 representative of 90 percentile are displayed. Data for displaying statistical growth curves 204 and 206 are provided from mother-child database server 6 in response to such a series of operations at mobile phone 4 that ID box 210, password box 212, handle box 214 and gender box 216 are all filled and send button 218 is operated by means of operating portion 26 to access mother-child database server 6 and upload the newest weight data corresponding to plot 208 on the graph.

Alternatively, the user can simply make an operation to display growth chart by means of operating portion 26 if the user would not desire to send the newest weight data corresponding to plot 208 to mother-child database server 6 for the reason of privacy protection. And, general data of growth curves 204 and 206 for 10 percentile and 90 percentile on the basis of the latest year statistics, which has been downloaded with the growth chart software upon the initial register with the ID and password, is displayed on display 28 with growth curve 202 on the basis of data stored in memory 24 displayed in comparison with general growth curves 204 and 206. Thus, the display of growth chart at display 8 as shown in FIG. 3 can be made without any communication with mother-child database server 6 on the occasion of displaying the newest weight data corresponding to plot 208 on the graph.

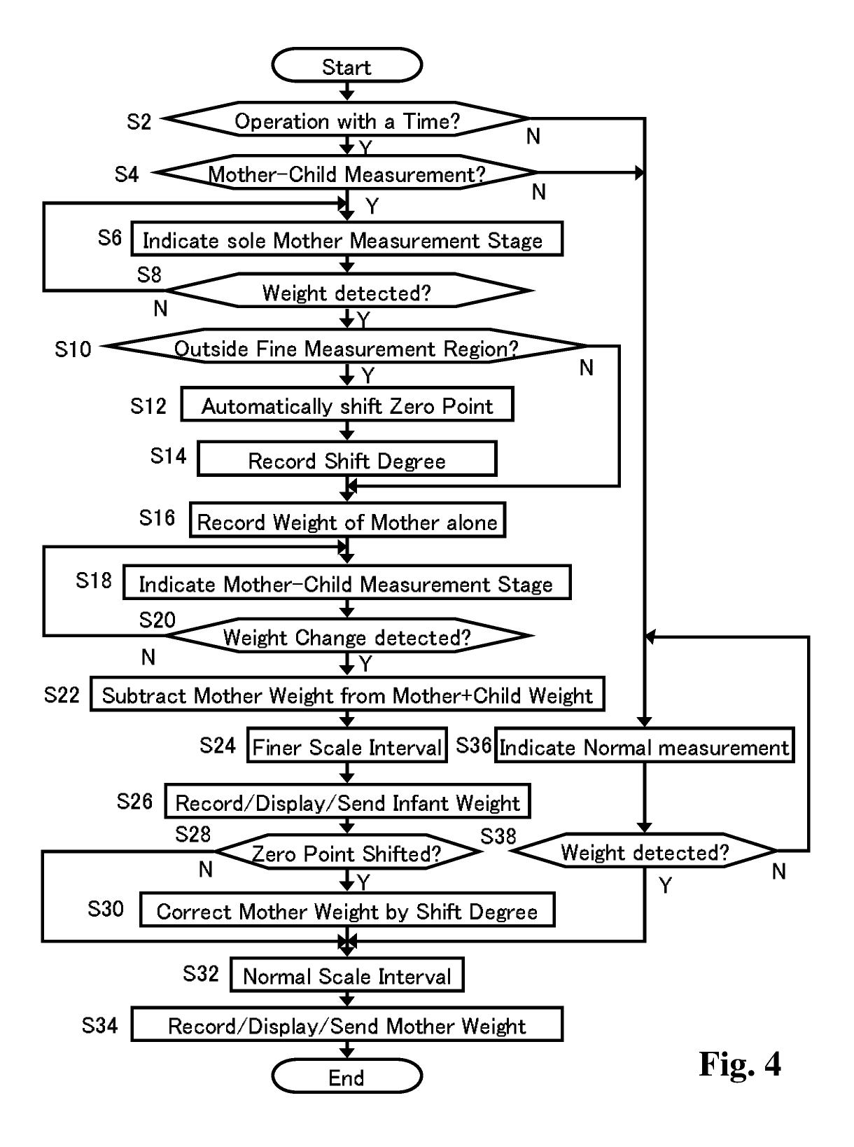

FIG. 4 is a flowchart showing the function carried out by scale controller 8 of electronic scale 2 in FIG. 1. The flowchart starts when electronic scale 2 is powered on, and it is checked in step S2 whether or not any operation relating measurement is done within a predetermined time from the power on of electronic scale 2. If the operation is detected in step 2, the flow goes to step S4 to check whether or not the operation is for mother-child measurement. If it is the case of mother-child measurement operation, the flow goes to step S6 to indicate on display 16 that the stage is for measuring mother's weight on her own, the flow then going to step S8. In step S8, it is checked whether or not weight sensor 12 detects any weight to return to step S6 upon no detection. Thus, steps S6 and S8 are repeated to wait a detection of weight with the indication of the mother measurement stage continued.

If it is determined in step S8 that weight sensor 12 detects some weight, the flow goes to step S10 to check whether or not the detected weight of mother alone is outside the high sensitivity measurement region, or the fine measurement region. As has been mentioned, the case of mother's weight outside the fine measurement region corresponds to a first situation that the weight of mother alone dips from the lower end of the fine measurement region or a second situation that the weight of mother plus a supposed infant is expected to outweigh the upper end of the fine measurement region due to the weight of mother alone. If it is determined in step S10 that mother's weight is outside the fine measurement region, the flow goes to step S12 to alter the zero point to shift the entire dynamic range in parallel so that the fine measurement region may become to cover both the weight of mother alone and the weight of mother plus a supposed infant. The degree or amount of the shift is recorded is step S14, the flow going to step 16. On the other hand, if it is not determined in step S10 that mother's weight is outside the fine measurement region, the flow directly goes to step S16, any zero point shift being not made in this case.

In step S16, the weight of mother alone measured in accordance with the process above is recorded in memory 10. Next in step S18 it is indicated at display 16 that the stage is advanced for measuring weight of mother plus infant, the flow then going to step S20. In step S20, it is checked whether or not weight sensor 12 detects any change in weight due to the infant taken in mother's arms to return to step S6 upon no detection. Thus, steps S18 and S20 are repeated to wait a detection of change in weight with the indication of the mother-child measurement stage continued. Step 20 can detect the change both in a first case that mother takes over infant with herself kept on electric scale 2 and in a second case that mother once got off electronic scale 2 and steps thereon again with infant in her arms by means of the comparison with the weight of mother alone having been recorded in step S16.

If it is determined in step S20 that weight sensor 12 detects some change in weight, the flow goes to step S22 to calculate the weight of infant alone by means of subtracting the weight of mother alone recorded in step S16 from the weight of mother plus infant detected in step S20. Here, it should be noted that the weight of mother plus infant used in calculation in step S22 is the weight measured with zero point shifted if the zero point shift has been made in step S12, the amount of zero point shift in the measurement in step S20 for the weight of mother plus infant in this case being identical with that in the record in step 16 for the weight of mother alone, which are exactly cancelled through the subtraction in step S22. Next in step S24, the finer scale interval is adopted to record the infant weight in memory 10 and display it at display 16 in step S26. Further in step S26, the infant weight stored in memory 10 is automatically sent to mobile phone 4 by way of scale short-range communicator 18 and mobile phone short-range communicator 20. As long as the process of steps S16 to S26 above for treating the weight of infant, the process is not to do with whether or not zero point shift is carried out in step S12.

In contrast the above, the process led by step S28 for treating the weight of mother closely depend on the fact whether or not zero point shift is carried out in step S12. So, it is checked in step S28 whether or not the zero point adjustment has been made in step S12. If it is determined in step S28 that the zero point shift has been made in step S12, the flow goes to step S30 to correct the weight of mother alone recorded in step 16 by the shift degree recorded in step S14, the flow then going to step S32. On the other hand, if it is not determined in step S28 that the zero point shift has been made in step S12, the flow directly goes to step S32, because any correction is naturally unnecessary in this case. In step S32, the normal scale interval is adopted in place of the finer scale interval to record the mother weight in memory 10 and display it at display 16 in step S34. Further in step S34, the mother weight stored in memory 10 is automatically sent to mobile phone 4 by way of scale short-range communicator 18 and mobile phone short-range communicator 20, the flow then going to the end. The mother weight sent to mobile phone 4 is processed to diagnose whether or not the mother is doing well after childbirth by means of the mother-child notebook software.

By the way, if it is not determined in step S2 that any operation relating measurement is done within a predetermined time from the power on of electronic scale 2, the flow goes to step S36 to indicate on display 28 that electronic scale 2 is automatically set into normal measurement status. Similarly, if it is determined that the operation made within the predetermined time is not for mother-child measurement, the flow goes to step S36 for normal measurement. Next in step S38, it is checked whether or not weight sensor 12 detects any weight to return to step S36 upon no detection. Thus, steps S36 and S38 are repeated to wait a detection of weight with the indication of the normal measurement status continued. If it is determined in step S38 that weight sensor 12 detects some weight, the flow goes to step S32 to adopt the normal scale interval and to record the weight in memory 10, to display it at display 16 and to send it to mobile phone 4 in step S34, the flow then going to the end also in such a normal measurement case. In the above explanation, it is the mother who measures her weight in the normal measurement case. However, not only the mother, but also any person such as another member of the family can measure its weight in the normal measurement case. So, with respect to the normal measurement case, it should be noted that step S34 is understood to read “Record/Display/Send weight of a person” or the like.

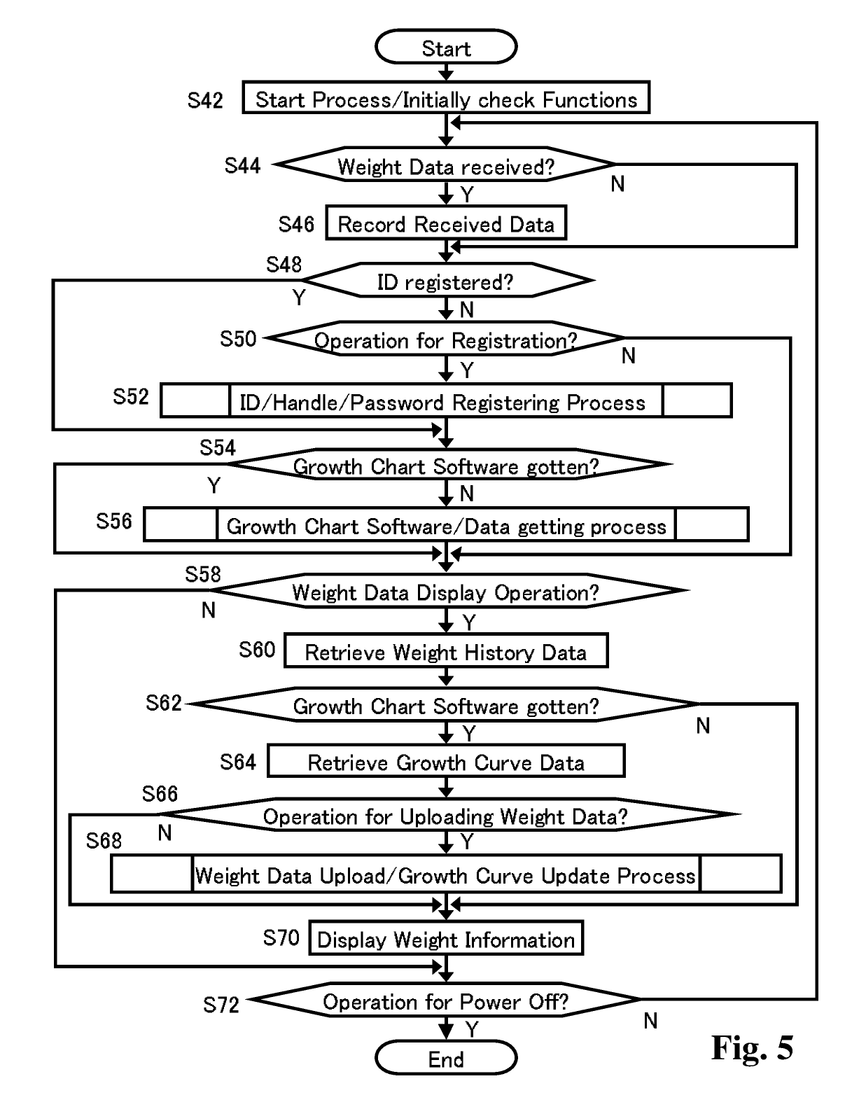

FIG. 5 is a flowchart showing a partial function carried out by mobile phone controller 22 of mobile phone 4 in FIG. 1 in relation with mother-child notebook software. The flowchart starts when mobile phone 4 is powered on to go to step S42, in which it is directed to start process and to initially check various functions of mobile phone 4. Next, in step S44, it is checked whether or not any new weight data is received from electronic scale 2. If it is the case, the flow goes to step S46 to record the weight data into memory 24, the flow the going to step S48. On the other hand, if it is not determined that any new weight data is received from electronic scale 2, the flow directly goes to step S48.

In step S48, it is checked whether or not the mother-child notebook ID has been registered. As has been mentioned, the mother can be informed of the ID upon provision of the mother-child notebook software under a strict personal information protection procedure. This is not only for protecting privacy of mother and child, but also for increasing the reliability of statistics by mother-child database server 6 against malicious attacks or false weight data upload. The registration of the ID is at mother's option.

If it is determined in step S48 that the mother-child notebook ID has not been registered, the flow goes to step S50 to check whether or not an operation for registering ID at operation portion 26 is made. If such an operation is detected in step 50, the flow goes to step S52 for accessing mother-child database server 6 to carry out a series of registration process in accordance with instructions by mother-child database server 6, the flow then going to step S54. The process carried out in step S52 includes authentication of the ID and the tentative password for initial entry into mother-child database server 6 and the registration of ID, the handle and the formal password in preparation for the next access. On the other hand, if it is determined in step S48 that the mother-child notebook ID has already been registered in mother-child database server 6, the flow directly goes to step S54.

In step S54, it is checked whether or not the growth chart software has been gotten. If not, the flow goes to step S56 for accessing mother-child database server 6 to download and install the growth chart software with general data of growth curves for 10 percentile and 90 percentile on the basis of the latest year statistics, which is necessary for mobile phone 4 to make display as in FIG. 3, the flow then going to step S58. On the other hand, if it is determined in step S54 that the growth chart software has been gotten, the flow directly goes to step S58. Further, if it is not determined in step S50 that the operation for registering ID is made, the flow directly goes to step S58.

In step S58, it is checked whether or not an operation for displaying weight data is made at operation portion 26. If such an operation is detected in step S58, the flow goes to step S60 to retrieve weight history data from memory 24. And, in step S62, it is checked whether or not the growth chart software has been gotten. If it is the case, the flow goes to step S64 to retrieve the data of growth curves for 10 percentile and 90 percentile from memory 24. Further, it is checked in step S66 whether or not an operation is made at operation portion 26 to upload the private infant weight data in memory 24 to mother-child database server 6. If such an operation is detected in step S66, the flow goes to step S68 for accessing mother-child database server 6 to upload the private infant weight data and, in exchange for it, to download the data of updated growth curves for 10 percentile and 90 percentile with the uploaded data incorporated for updating memory 24, the flow going to step S70. The above mentioned process in step 68 corresponds to the explanation in conjugation with FIG. 3.

On the other hand, if it is not determined in step S66 that the operation to upload the private infant weight data, the flow directly goes to step S70. Further, if it is not determined in step S62 that the growth chart software has been gotten, the flow directly goes to step S70. In step S70, weight information in accordance with the previous steps is displayed. In other words, if step S70 is reached by way of step S68, growth chart as in FIG. 3 is displayed with the statistics growth curves 204 and 206 for 10 percentile and 90 percentile displayed with personal growth curve 202 being of the updated ones. On the other hand, if step S70 is reached directly from step S66, the statistics growth curves 204 and 206 in growth chart in FIG. 3 are on the basis of the latest year statistics. Further, if step S70 is reached directly from step S62, only personal growth curve 202 with weight data stored in memory 24 simply plotted is displayed.

The display by the function of step S70 is automatically continued until a predetermined time has lapsed. With the automatic termination of the display, the flow goes to step S72 to check whether or not an operation is made at operation portion 26 to turn off the power supply to mobile phone 4. If such an operation is detected in step S72, the flow goes to the end. Further, if it is not determined in step S58 that the operation for displaying weight data is made at operation portion 26, the flow goes to step S 72 for checking the power off operation. If the power off operation is not detected in step S72, the flow returns to step S44 to repeat steps S44 to S72 unless the power off operation is detected in step S72.

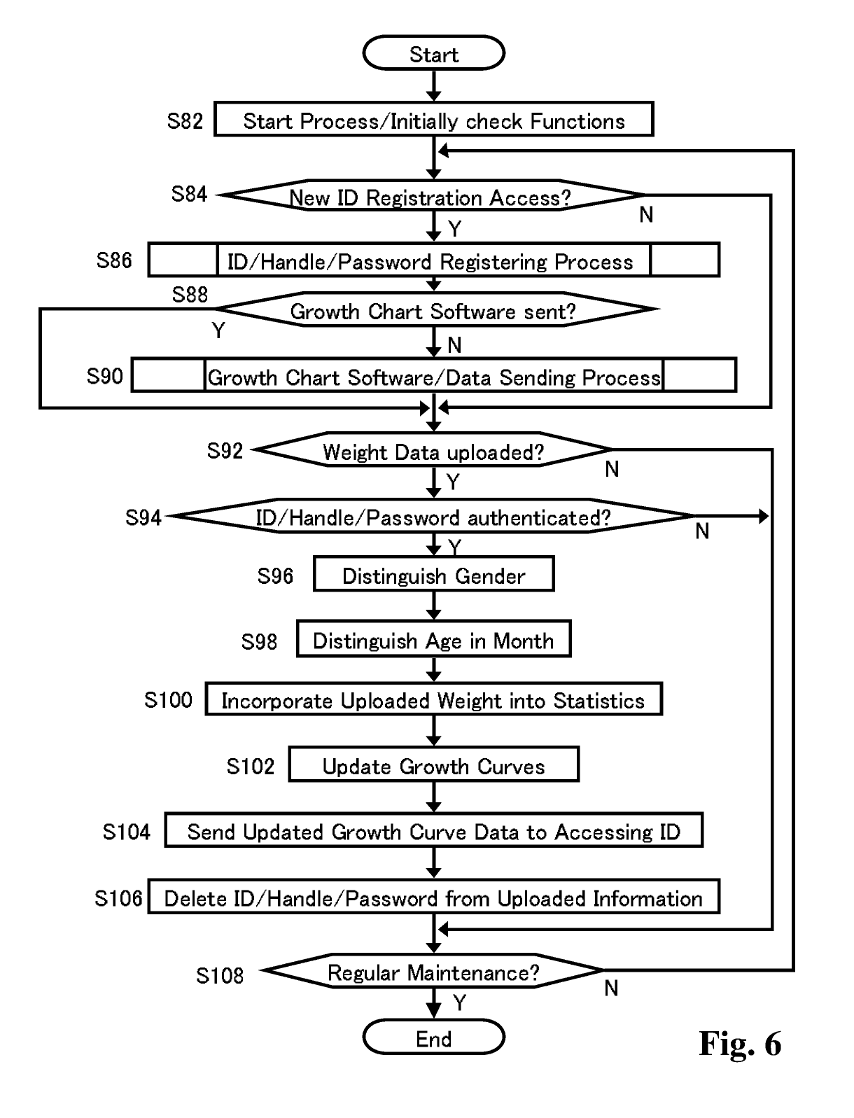

FIG. 6 is a flowchart showing the mother-child notebook software function carried out by server controller 34 in FIG. 1. The flowchart starts when server controller 34 is started up to go to step S82, in which it is directed to start process and to initially check various functions of mother-child database server 6. Next, in step S84, it is checked whether or not any new access for ID registration on the mother-child notebook system. If the access is detected in step 84, the flow goes to step S86, in which a series of registration process is carried out in communication with mobile phone 4 accessing to mother-child database server 6, the flow then going to step S54. The process carried out in step S86 includes authentication of the ID and the tentative password from the accessing mobile phone 4 and the registration of ID, the handle and the formal password in preparation for the next access.

Next in step S88, it is checked whether or not the growth chart software has been sent to the accessing mobile phone 4. If not, the flow goes to step S90 for sending to mobile phone 4 the growth chart software with general data of growth curves for 10 percentile and 90 percentile on the basis of the latest year statistics, the flow then going to step 92. On the other hand, if it is determined in step S88 that the growth chart software has been sent, the flow directly goes to step S92. Further, if the new ID registration access operation is not detected step S84, the flow directly goes to step S92.

The steps led by step S92 are for updating the statistics of growth curves for 10 percentile and 90 percentile with an uploaded new weight data incorporated. In step S92 it is checked whether or not a new weight data is uploaded. If a new data upload is detected in step S92, the flow goes to step S94 to authenticate ID, handle and password for fear of malicious attacks or false weight data upload. If the authentication is successful in step S94, the gender of the infant is distinguished in step S96, and the age in month of the infant in step S98 to incorporate the uploaded weight data of the infant into the statistics of growth chart based on gender and age in month in step S100. By means of the preceding steps, the growth curves for one of the genders corresponding to that of the infant is updated in step S102. Thus, the updating of is carried out every time when new weight data is uploaded in the case of the flowchart of FIG. 6. However, in place of such a real time update, the update step such as step 102 may be carried out every 24 hours in a lump sum of data accumulated for one day.

Next in step 104, the updated data of growth curves is sent to ID authenticated in step S94. Further in step S106, private information related to the weight data uploaded, such as ID, handle and password, are deleted for keep the privacy of mother and infant, the flow then going to step S108. On the other hand, if no upload of new weight date is detected in step S92, the flow directly goes to step S108. Also, the authentication is failed in step S94, the flow goes to step S108. In step S108 it is checked whether or not it is the time for regular maintenance of mother-child data base server 6 to go to the end if it is the case. On the other hand, if it is determined in step 108 that the regular maintenance time has not come, the flow goes back to step S84 to repeat steps S84 to step S108 till the regular maintenance time comes.

The various features set forth above are not limited to the application to the explained embodiment, but is widely applicable to other embodiments. For example, the features relating to the electronic mother-child notebook are advantageous not only in combination with electronic scale 2 of Embodiment 1 which measures mother and infant weight, but also in combination with a general scale for sole purpose of measuring infant weight. Further, in place of Embodiment 1, in which electronic scale 2 is separated from mobile phone 4, all functions relating to the electronic mother-child notebook are incorporated into electronic scale 2 so as to form an integrated electronic scale having electronic mother-child note book functions. In this case, electronic scale directly communicates with mother-child database 6. By the way, the features of electronic scale 2 in Embodiment 1 are advantageous not only in measuring the weight of mother and infant, but also in measuring the weight of a pet animal in such a manner that the animal guardian steps on electronic scale with the hyperactive pet animal held in his or her arms. Further, the features of electronic scale 2 in Embodiment 1 are advantageous in knowing the difference in weight of the same person in a finer scale interval which may change as a result of diet plan.

### Embodiment 2

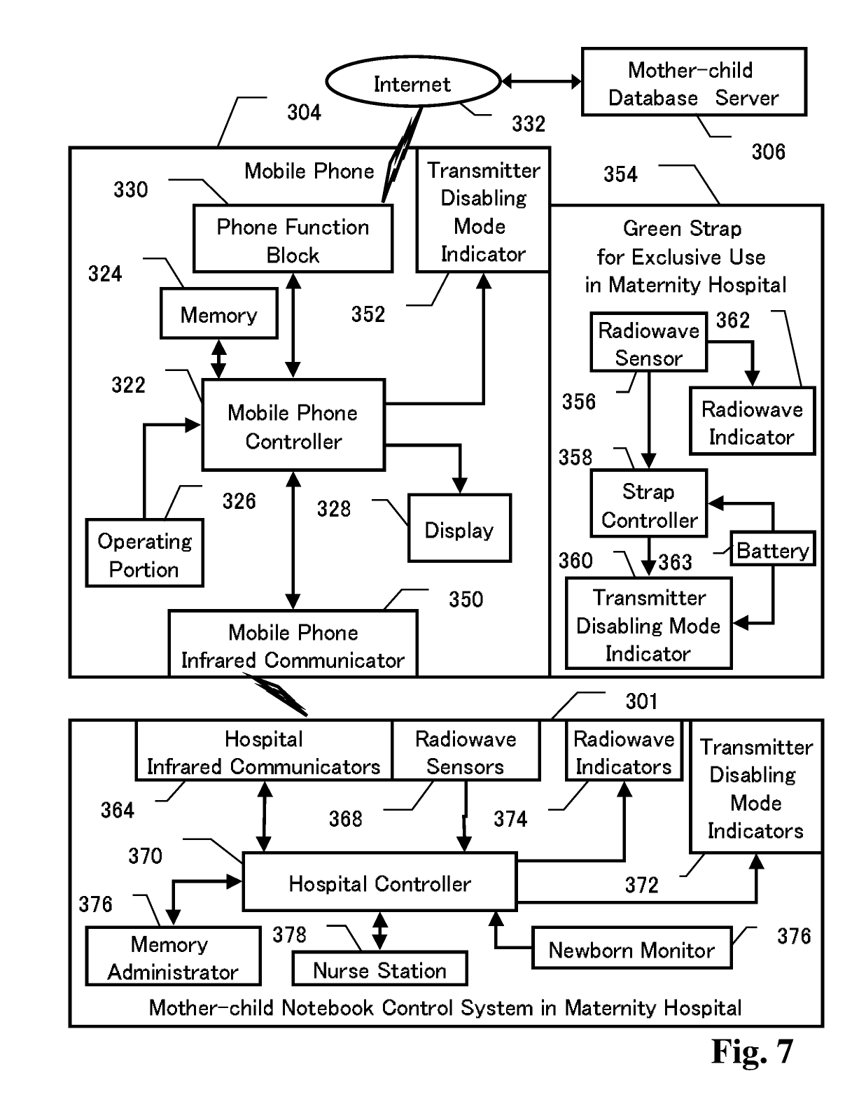

FIG. 7 is a block diagram showing the mother-child database system according to Embodiment 2 of this invention, elements of which are all numbered with three hundreds. The system in Embodiment 2 includes mother-child notebook control system 301 in maternity hospital, mobile phone 304 and mother-child database server 306. Most of the details in mobile phone 304 are similar to those of Embodiment 1 in FIG. 1 with numbers of the identical lower two digits given, respectively, the explanations of which are accordingly omitted except for the case of necessity. For example, mobile phone controller 322 in FIG. 7 is similar to mobile phone controller 22 in FIG. 1. Mobile phone 304 of Embodiment 2, in contrast to mobile phone 4 in Embodiment 1, is characterized by mobile phone infrared communicator 350 and transmitter disabling mode indicator 352 for informing surrounding people of mobile phone 304 functioning in transmitter disabling mode. The infrared communication by means of infrared light is carried out, for example, by IrDA system.

Mobile phones in general are known to transmit radiowave not only in phone conversation but also in standby state waiting a call, which radiowave is inhibited in an airplane or in hospital or the like not to cause a harmful influence on neighboring electronics. On the other hand, mobile phones are capable of various functions needless to rely on radiowave, such as a music player function or a camera function. The transmitter disabling mode is provided for a user of such a mobile phone to enjoy the transmission free functions above by means of disabling radiowave transmission with the main power itself kept in active state. For the purpose of avoiding problems, transmitter disabling mode indicator 352 is provided to inform surrounding people that mobile phone 304 is functioning in transmitter disabling mode. In addition to the information of the transmitter disabling mode by display 328, transmitter disabling mode indicator 352 is arranged to emit remarkable and meaning-acknowledged unique light, such as blinking green light, to the outside of mobile phone 304 so that the surrounding people may more easily notice the transmitter disabling mode. By the way, transmitter disabling mode indicator 352 and mobile phone infrared communicator 350 in FIG. 7 can be adopted in Embodiment 1 in FIG. 1. Similarly, mobile phone short-range communicator 20 in FIG. 1 can be adopted in Embodiment 2 in FIG. 7.

As in Embodiment 1, mobile phone 304 of Embodiment 2 in FIG. 2 is capable of recording and displaying various data relating to the growth of infant such as body height and circumference of head as well as the prehistory data during pregnancy. Also as in Embodiment 1, these functions are controlled by mother-child notebook software installed in memory 324. Thus mobile phone 304 serves as electronic mother-child notebook as in Embodiment 1.

As in Embodiment 1, phone function block 330 makes it possible for mobile phone 304 to communicate with mother-child database server 306 by way of internet 332. As in Embodiment 1, mother-child database server 306 also serves as an online portal for free information service in cooperation with healthcare authority and baby item industry. Thus, mobile phone 304 with the ID and password registered can login and receive from mother-child database server 306 a growth chart software with general data of growth curves for 10 percentile and 90 percentile on the basis of the latest year statistics as in Embodiment 1.

Further, if mobile phone 304 logins with the ID and password to upload under anonymity the newest weight data in memory 324 for own baby, mother-child database server 306 provides mobile phone 304 with special data of growth curves for 10 percentile and 90 percentile created by means of the statistic on the basis of the newest data currently uploaded under the same condition from a great number of mobile phones including mobile phone 304. This function is also similar to that in Embodiment 1.

As has been clear from the above, Embodiment 2 in FIG. 7 includes a function relating to the electronic mother-child notebook basically similar to that in Embodiment 1. Embodiment 2 is further characterized by an additional function useful for a mother in several days in maternity hospital directly after giving birth. A mother directly after giving birth is advised by a doctor or a nurse to regularly record and report several times in one day the condition of the newborn baby, such as the body temperature, the defecation and/or urination with its approximately estimated amount. Also the mother is advised to record and report the frequency and amount of breast-feed with the observation of appetite and with or without milk disgorgement thereafter. Conventional manner of such record and report is fulfilled by means of a detailed paper form prepared by the maternity hospital. In contrast to the conventional manner of the paper record and report, Embodiment 2 makes it possible for the mother to carry out the record and report by means of mobile phone 304 with the mother-child notebook software installed in memory 324, which includes in-hospital report mode.

As in other hospitals, maternity hospitals basically inhibit the use of mobile phone and request the visitor to shut off the main switch of the mobile phone. Embodiment 2, however, proposes to use mobile phone in a maternity hospital to make use the function of electronic mother-child notebook in the in-hospital report mode. For this purpose, mobile phone 304 in Embodiment 2 is arranged be set into the transmitter disabled mode when electronic mother-child not book software functions. However, two problems are caused here. The first problem is how to make it sure that mobile phone 304 is set into the transmitter disabled mode without fail as long as the electronic mother-child not book software functions. The second problem is how to avoid trouble in advance by means of surely informing the surrounding people of the transmitter disabling mode setting.

To solve the first problem, mobile phone 304 of Embodiment 2 is arranged to forcibly and automatically change into the transmitter disabling mode without fail if an operation for the mother-child notebook function in in-hospital report mode is made. On the other hand, mobile phone 304 is so arranged that the in-hospital mode is inhibited during the use of the mother-child notebook function unless the transmitter disabling mode is set. The details of the above arrangement will be explained later. As to the second problem, the above mentioned transmitter disabling mode indicator 352 is one of the solutions of the second problem. Embodiment 2 is provided with a further solution of the second problem as will be explained below.

To solve the second problem relating to notification of the transmitter disabling mode, the system according to Embodiment 2 includes green strap 354 for exclusive use in the maternity hospital, which is lent by the maternity hospital to a mother who has registered mobile phone 304 with the mother-child notebook software installed for use in the maternity hospital. The mother is obliged to attach green strap 354 to mobile phone 354. Green strap 354 is of a unique shape, the meaning of which is well acknowledged within the maternity hospital, and of green appearance symbolizing safety.

Further, Green strap 354 includes radiowave sensor 356. Strap controller 358 controls transmitter disabling mode indicator 360 to emit indication light as long as radiowave sensor 356 does not detect the radiowave. The color of the indication light from transmitter disabling mode indicator 360 is also green symbolizing safety. It should be noted that indication is positively made by transmitter disabling mode indicator 360 when the output of radiowave sensor 356 is negative. Thus, by means of normally making indication of safety, notification effect is increased for the purpose of removing misunderstanding of the surrounding people, which otherwise may cause troubles. For the purpose of the normal indication of safety, green strap 354 include button battery 363 an own power source. Radiowave indicator 362 is powered by the electromotive force of radiowave sensor 356 excited by the radiowave form mobile phone 304 to indicate radiowave transmission even if the button battery 363 has been exhausted, the radiowave being caused by some wrong operation. The indication light from radiowave indicator 362 is of red color symbolizing caution and blinking to cause perceivable change. Radiowave indicator 362 of course makes radiowave indication by the electromotive force of radiowave sensor 356 if button buttery 363 is sufficient. Thus, the mother can instantly shut off the main switch of mobile phone 304 or set mobile phone 304 into the transmitter disabling mode if she notices the red blinking light from green strap. If mobile phone 304 is carried out of the maternity hospital to make conventional phone conversation or data communication through the Internet, green strap 354 is to be removed from mobile phone 304 and retrieved by the maternity hospital for avoiding unnecessary confusion.

Under the above circumstance with the acknowledgement by the surrounding people, the mother in the maternity hospital inputs the record of body temperature, the defecation and/or urination, breast-feed and milk disgorgement, or the like into mobile phone 304 with the mother-child notebook software set to the in-hospital report mode. The input is possible through the graphical user interface (GUI) on display 328 in cooperation with operating portion 326. For example, mother-child notebook software presents an input windows on display 328 for items of body temperature, defecation and/or urination, breast-feed and milk disgorgement, or the like for easy selection or input by means of keys or the like in operating portion 326.

On the other hand, mother-child notebook control system 301 in maternity hospital prepares many hospital infrared communicators 364 at various convenient key points such as each patient room or each bedside available to each mother. Further, radiowave sensors 368 are prepared each located adjacently to each of hospital infrared communicators 364. Thus, the data input into mobile phone 304 is to be reported to maternity hospital by way of the communication between mobile phone infrared communicator 350 and hospital infrared communicator. Hospital controller 370 controls transmitter disabling mode indicator 372 to emit green indication light as long as radiowave sensor 368 does not detect the radiowave. Thus, surrounding people are informed of safety to feel easy even if a mother operates mobile phone 304 for reporting the newborn data to maternity hospital through infrared communication. Transmitter disabling mode indicator may arranged to further display or announce message such as “Safe data transmission with radio wave transmitter disabled”, or the like.

On the contrary, if one of radiowave sensors 368 detects radiowave caused by some wrong operation, corresponding one of transmitter disabling mode indicators 372 ceases the indication and corresponding one of radiowave indicators 372 starts to blink with red light. The red light blinking is mainly for making a caution to the mother herself near the corresponding one of the convenient key points to instantly shut off the main switch of mobile phone 304 or set mobile phone 304 into transmitter disabling mode. It is needless to say that radiowave indicator 362 of green strap 354 firstly starts to blink in red in the above case of erroneous radiowave transmission from mobile phone 304 regardless of the place where mobile phone 304 exists in the maternity hospital to make the caution.

In mother-child notebook control system 301 in maternity hospital, memory administrator 376 takes on the basic function under control of hospital controller 370. According to the basic function, memory administrator 376 stores and administrates the report data from mobile phone 304 or the like through hospital infrared communicators 364. Mother-child note book control system 301 in maternity hospital also administrates newborns in maternity hospital. For example, newborn monitor 376 monitors existence/nonexistence or movement or cardiac sound of newborns in nursery room to automatically input the results into hospital controller 370. The data from newborn monitor 376 as well as the report originated by mobile phone and stored in memory administrator 376 is transmitted to nurse station 378, in which the transmitted data are displayed on monitor screen in nurse station for watching by nurses.

The infrared communication by means of mobile phone infrared communicator 350 and hospital infrared communicator 364 is bidirectional. In other words, not only the one way of data transmission from mobile phone 304 to mother-child notebook control system 301, the other way from mother-child notebook control system 301 to mobile phone 304 is possible for the purpose of transmitting date gotten by newborn monitor 376 to mobile phone display 328 or informing mobile phone 304 of comment and advice from nurse station 378.

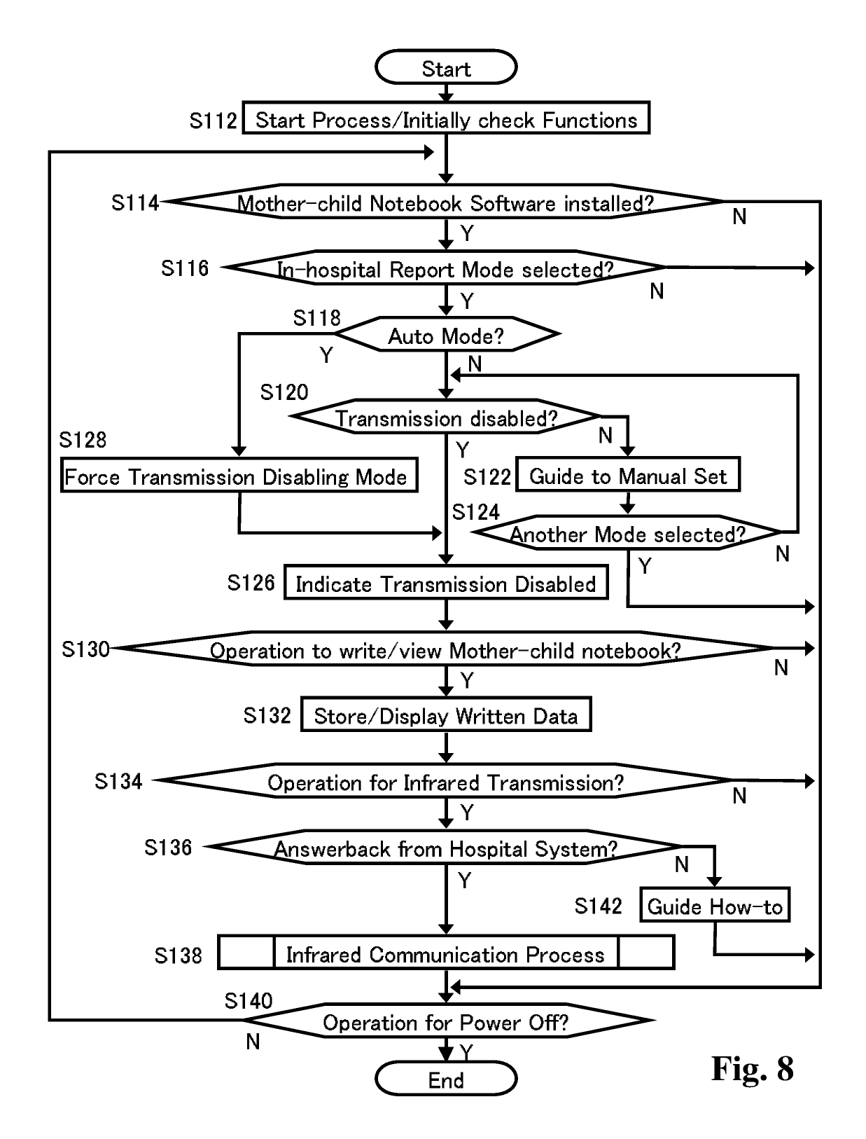

FIG. 8 is a flowchart showing a partial function carried out by mobile phone controller 322 of mobile phone 304 in FIG. 7 in relation with mother-child notebook software. The flowchart starts when mobile phone 304 is powered on to go to step S82, in which it is directed to start process and to initially check various functions of mobile phone 304. Next in step S114 it is checked whether or not the mother-child notebook software has been installed. If it is determined in step 114 that the software has been installed, the flow goes to step S116 to check whether or not in-hospital report mode is selected. And, if it is determined in step S116 that the mode is selected, the flow goes to step S118. On the contrary, if the mother-child notebook software is not used in the in-hospital report mode but is used at home as in such a case that weight of the baby is scaled and recorded, step S116 never advance to step S118.

In step S118, it is checked whether or not the automatic mode is set. The automatic mode is for automatically forcing the transmission disabling mode, the automatic mode being set in advance upon custom setting of mobile phone 304. If automatic mode setting is not determined in step S118, it means that mobile phone 304 is in the manual mode. Thus, flow goes from step S118 to step S120 in this case to check whether or not the transmission disabling mode has been set. If not, the flow goes to step S122 to make a guidance by announce or message on display 328 to manually set the transmitter disabling mode. Further, in step S124, it is checked whether or not a mode other than the mother-child mote book mode or another mode within the mother-child mote book mode is selected. If not, the flow returns to step S120. Thus, unless the mode setting above is not done, the loop of step 120 to step S124 is repeated to wait for detection of the transmitter disabling mode setting. In other words, unless the transmitter disabling mode is manually set, the flow is never advanced to step S126 which leads the transmitter disabling mode.

If it is determined in step S118 that the automatic mode is set, the flow goes to step S128 to automatically force the transmitter disabling mode, the flow then advancing to step S126 as in the case that the transmitter disabling mode is confirmed in step S120. It should be noted that flow goes from step S118 to step S126 by way of step S128 with no function in the case that the transmitter disabling mode has been set at the stage of step S118. Thus, in the automatic mode, the transmitter disabling mode is set in automatic and instant response to the in-hospital report mode selection at mobile phone 304, which is detected in step S116. As is apparent from the above explanation, step S126 is never reached unless mobile phone 304 is set to the transmitter disabling mode regardless of whether the setting is done automatically or manually. In this way, the in-hospital report mode in the mother-child notebook mode is assured to be carried out in the transmitter disabling mode without fail.

In step S126, it is indicated at transmitter disabling mode indicator 352 that mobile phone 304 is functioning in the transmitter disabling mode for notice to surrounding people. The indication of the transmitter disabling mode is also made at display 328, which is for informing the mother herself of the condition of mobile phone 304. The flow advances to step S130 with the indication of the transmitter disabling mode continued.

In step 130, it is checked whether or not an operation is made for writing a new report or viewing a former report. If a relevant operation is detected in step S130, the flow goes to step S132 to store the new report and display the written data. In the case that only viewing operation is detected in step S130, the former report is displayed in step S132. With the display continued, the flow advances to step S134 to check whether or not an operation is made for infrared transmission of the newly written report to mother-child notebook control system 301 in maternity hospital. If the transmission operation is detected in step S134, the flow goes to step S136 to check whether or not an answerback code comes from mother-child notebook control system 301 in maternity hospital. If the answerback code is detected in step S136, the flow goes to step S138 to carry out infrared communication process, in which the newly written report is transmitted to and necessary feedback data is received from mother-child notebook control system 301 in maternity hospital, the flow then going to step S140. Since the carry out infrared communication process in step S138 is carried out with mobile phone 304 identified by means of ID, mother-child notebook control system 301 in maternity hospital distinguish the sender of the report. On the other hand, if mobile phone 304 fails to receive any answerback in step S136, the flow goes to step S142 to make a guidance how to deal with such a situation, the flow then going to step S140. The guidance made in step S142 includes an explanation of suitable correct operation required by mother-child notebook control system 301 in the case of some wrong operation causing the lack of the answerback, and a recommendation of move to some suitable convenient key point in the case of transmission operation at an improper point causing to lack of the answerback.

If it is not determined in step 114 that the mother-child notebook software has been installed, the flow directly goes to step S140. Similarly, if it is not determined in step S116 that the in-hospital report mode is selected, or if it is determined in step S124 that a mode other than the mother-child mote book mode or another mode within the mother-child mote book mode is selected, or if the operation to write-in or view mother-child notebook is not detected in step 130, or the operation for infrared transmission is not detected in step S134, the flow directly goes to step S140.

In step S140 it is checked whether or not an operation is made at operation portion 326 to turn off the power supply to mobile phone 304. If such an operation is detected in step S140, the flow goes to the end. On the contrary, if the power off operation is not detected in step S140, the flow returns to step S114 to repeat steps S114 to S142 unless the power off operation is detected in step S140.

The various features of the present invention relating to the input and report of newborn information described in Embodiment 2 are not necessarily limited to the details explained above, but are widely applicable to other embodiments. For example, the input newborn information may be transmitted to another mobile phone held by a nurse through infrared communication if the mother-child notebook software is also installed in the mobile phone of the nurse. In this case, it is not necessary to set up a special mother-child notebook control system 301 in the maternity hospital, but the mother-child notebook system can be instantly established by installing the mother-child notebook software in mobile phones which nurses or mothers conventionally use. It is needless to say that green strap 354 has to be attached to each of the mobile phones which nurses hold.

According to another modification of Embodiment 2, the input of the newborn is not necessarily carried out by mobile phone 304, but may be carried out by a special input device with an operating portion and a display for GUI prepared by the maternity hospital at each bedside. In this case the transmission of the input newborn from the input device to mother-child notebook control system may not necessarily carried out by wireless communication, but wired communication through cable network in the maternity hospital, which inherently free from the problem of radiowave generation. Even in such a case the combination of transmitter disabling mode indicator 352 in mobile phone 304 with the mother-child notebook software installed and green strap 354 is useful. This is because that mobile phone 304 may be necessitated to download through infrared communication in the maternity hospital the newborn data history initially input by the special input device and stored in memory administrator 376. If a mother has been accumulating data in a medium-term from beginning pregnancy to the babyhood, the data download in the maternity hospital under the transmission disabling mode explained above is extremely advantageous.

The various features of the present invention relating to the green strap 354 of Embodiment 2 is not necessarily limited to the mother-child notebook system in the maternity hospital, but are widely applicable to other embodiments. For example, the green strap may not be limited to the exclusive use in the maternity hospital, but similar green strap may be applicable to the case of general hospitals as long as the meaning of the green strap is locally acknowledged by all people in the hospitals. Further, if a notice of the meaning of the information means such as green strap indicative of the transmitter disabling mode is thoroughly made in advance among all members within a special area and the popularization and the administration of the information means within the area is strict, the special area may not necessarily be limited to hospitals, but may be any local facilities or areas other than hospitals.

Further, the wireless communicator to be used in the transmitter disabling mode is not necessarily limited to the infrared communicator as in Embodiment 2, but other various suitable communicators may be widely adoptable. For example, the short-range communicator 18 and 20 using with weak radiowave in Embodiment 1 is adoptable if the strength of the radiowave is sufficiently less than a permissible level determined by the hospitals or other local area. Other examples of useable wireless communicator is Wireless Local Area Network, the faint signal radiowave communicator, Bluetooth, or the like. Human body surface communication system is another example of the communicator useable in the transmitter disabling mode.

What is claimed is:

1. A mobile phone capable of communication in a local area comprising:
a radiowave transmitter configured to generate radiowave for telecommunication function:
a radiowave receiver configured to receive radiowave for the telecommunication function;
an infrared transmitter configured to generate infrared light for wireless communication within the local area;
an infrared receiver configured to receive infrared light for the wireless infrared communication within the local area;
a processor configured to process data to be transmitted by the infrared transmitter on the infrared light within the local area and data received by the infrared receiver on the infrared light within the local area in accordance with a software downloaded by the radiowave receiver; and
a memory configured to store the software downloaded by the radiowave receiver for the processor to process the data.

2. The mobile phone according to claim 1 further comprising a display configured to display the data to be transmitted by the infrared transmitter on the infrared light within the local area and the data received by the infrared receiver on the infrared light within the local area in accordance with the software downloaded by the radiowave receiver and stored in the memory.

3. The mobile phone according to claim 2, wherein the display is further configured to display the data processed by the processor in accordance with the software downloaded by the radiowave receiver and stored in the memory.

4. The mobile phone according to claim 1 further comprising a data input portion configured to input data to be transmitted by the infrared transmitter on the infrared light within the local area in accordance with the software downloaded by the radiowave receiver and stored in the memory

5. The mobile phone according to claim 4, wherein the display is further configured to display the data input by the data input portion in accordance with the software downloaded by the radiowave receiver and stored in the memory.

6. The mobile phone according to claim 1 further comprising a controller to download the software from an outside server through internet by means of the radiowave receiver and to install the downloaded software into the memory for the processor to process data be transmitted by the infrared transmitter on the infrared light within the local area and data received by the infrared receiver on the infrared light within the local area in accordance with the software.

7. The mobile phone according to claim 6, wherein the controller is configured to access the outside with ID and password to download the software for the processor to process data be transmitted by the infrared transmitter on the infrared light within the local area and data received by the infrared receiver on the infrared light within the local area in accordance with the software.

8. The mobile phone according to claim 1 wherein the software includes a guidance how to deal with such a situation that the infrared transmitter fails in the wireless communication within the local area.

9. The mobile phone according to claim 1 wherein the software includes an explanation of the infrared transmitter in case a failure of the infrared transmitter in the wireless communication within the local area.

10. The mobile phone according to claim 1 wherein the software includes a recommendation of correct operation for the infrared transmitter for the wireless communication within the local area.

11. A mobile phone capable of communication in a local area comprising:
a radiowave transmitter configured to generate radiowave for telecommunication function:
a radiowave receiver configured to receive radiowave for the telecommunication function;
an infrared transmitter configured to generate infrared light for wireless communication within the local area;
an infrared receiver configured to receive infrared light for the wireless infrared communication within the local area;
a controller to download a software from an outside server through internet by means of the radiowave receiver, the software being for processing data to be transmitted by the infrared transmitter on the infrared light within the local area and data received by the infrared receiver on the infrared light within the local area; and
a memory configured to store the software downloaded by the radiowave receiver.

12. The mobile phone according to claim 11, wherein the controller is configured to access the outside with ID and password to download the software.

13. The mobile phone according to claim 11 wherein the software includes a guidance how to deal with such a situation that the infrared transmitter fails in the wireless communication within the local area.

14. The mobile phone according to claim 11 wherein the software includes an explanation of the infrared transmitter in case a failure of the infrared transmitter in the wireless communication within the local area.

15. The mobile phone according to claim 11 wherein the software includes a recommendation of correct operation for the infrared transmitter for the wireless communication within the local area.

16. A mobile phone capable of communication in a local area comprising:
a radiowave transmitter configured to generate radiowave for telecommunication function:
a radiowave receiver configured to receive radiowave for the telecommunication function;
an infrared transmitter configured to generate infrared light for wireless communication within the local area;
an infrared receiver configured to receive infrared light for the wireless infrared communication within the local area;
a controller to download a software from an outside server through internet by means of the radiowave receiver, the software including a guidance how to deal with such a situation that the infrared transmitter fails in the wireless communication within the local area; and
a memory configured to store the software downloaded by the radiowave receiver.

17. The mobile phone according to claim 16, wherein the controller is configured to access the outside with ID and password to download the software.

18. The mobile phone according to claim 16 wherein the software includes an explanation of the infrared transmitter in case a failure of the infrared transmitter in the wireless communication within the local area.

19. The mobile phone according to claim 16 wherein the software includes a recommendation of correct operation for the infrared transmitter for the wireless communication within the local area.
